(19) United States
(12) Patent Application Publication
(10) Pub. No.: US 2007/0293902 A1
(43) Pub. Date: Dec. 20, 2007

(54) Method of interfacing a sensor lead and a cardiac rhythm management device

(75) Inventor: Scott Thomas Mazar, Woodbury, MN (US)

Correspondence Address: FISH & RICHARDSON P.C., P.O. BOX 1022, MINNEAPOLIS, MN, 55440-1022, US

(73) Assignee: Transoma Medical, Inc., St. Paul, MN (US)

(21) Appl. No.: 11/454,294

(22) Filed: Jun. 16, 2006

(51) Int. Cl. A61N 1/365

(52) U.S. Cl. 607/17

(57) ABSTRACT

Embodiments of the invention provide a device for interfacing a specialized lead for sensing a specific physiological parameter, other than standard pacing and sensing of an electrocardiogram signal, with a conventional CRM device. This produces a lead-based sensing system that can be use with any CRM device that is capable of reading an electrocardiogram signal. In one embodiment, a lead-based sensing system for use with any CRM device that is capable of reading an electrocardiogram signal comprises a sensor configured to be coupled to any CRM device by a lead, and to generate a signal associated with a physiological parameter of a patient other than an electrocardiogram signal; and sensor electronics connected to the lead to convert the signal associated with the physiological parameter other than the electrocardiogram signal into a converted signal that is readable by any CRM device that is capable of reading an electrocardiogram signal.

### CROSS-REFERENCES TO RELATED APPLICATIONS

NOT APPLICABLE

### BACKGROUND OF THE INVENTION

Cardiac rhythm management (CRM) devices are used to stimulate the heart with electrical impulses to cause the heart to contract and thus to pump blood throughout the body. CRM devices have been developed which respond to the patient's activity level to provide variable pacing rates that more closely approximate the individual requirements of a patient.

Cardiac pacing leads designed to sense parameters, other than merely sensing standard electrocardiogram signals and pacing the heart, are typically of special design and require a CRM having circuitry specially designed to work with the special lead. For example, a pacemaker lead with a temperature sensor is disclosed in U.S. Pat. No. 4,726,383; it requires implantation of a specific lead containing a thermistor transducer. A specialized lead with a pressure sensor or an accelerometer is disclosed in U.S. Pat. No. 4,666,617; again it requires a specific lead with a built-in sensor. U.S. Pat. No. 4,690,143 discloses a pacemaker having a lead which can generate electrical power piezoelectrically from the movement of the lead. Such a lead requires a piezoelectric element built in along the length of the lead.

The aforementioned designs require a specialized lead for sensing special parameters. This can prove problematic for the patient when taken in conjunction with the need for the specialized lead to be used with a pacemaker responsive to such parameters. This can often serve to greatly limit the types of CRM devices that can be used by the patient and can also increase the cost to the patient, in that a more expensive and specialized CRM device may be required. It is not unusual for patients to require or want replacement CRM devices, either because the batteries on their previous device have been expended or to gain new and improved features with the updated device.

### BRIEF SUMMARY OF THE INVENTION

Embodiments of the present invention provide a device for interfacing a specialized lead for sensing a specific physiological parameter, or many physiological parameters, other than standard pacing and sensing of an electrocardiogram signal, with a conventional CRM device. This produces a lead-based sensing system that can be use with any CRM device that is capable of reading an electrocardiogram signal.

An aspect of the present invention is directed to a lead-based sensing system for use with any CRM device that is capable of reading an electrocardiogram signal. The lead-based sensing system comprises a sensor configured to be coupled to any CRM device by a lead, and to generate a signal associated with a physiological parameter of a patient other than an electrocardiogram signal; and sensor electronics connected to the lead to convert the signal associated with the physiological parameter other than the electrocardiogram signal into a converted signal that is readable by any CRM device that is capable of reading an electrocardiogram signal.

In some embodiments, the sensor is configured to sense a signal associated with any physical parameter (e.g., pressure, temperature, pH, displacement, acceleration, voltage, current, frequency, period, strain, force, acoustical parameters, fluid flow rate, and blood-oxygen saturation). The sensor electronics comprise a sensor interface coupled with the sensor to pre-process the signal from the sensor; and a signal processing unit configured to convert the signal from the sensor to the processed signal that is readable by any CRM device that is capable of reading an electrocardiogram signal. The sensor electronics may further comprise a current regulator connected to a power source which regulates output voltages to be between minimum and maximum levels; and a capacitor coupled to the current regulator to store an electrical charge and power a circuit comprising the sensor electronics. The power source comprises pacing pulses from the CRM device.

In accordance with another aspect of the present invention, a circuit for interfacing a sensor to a lead for a CRM device comprises a current regulator connected to a power source which regulates output voltages to be between minimum and maximum levels; a capacitor coupled to the current regulator to store an electrical charge and power the circuit; a sensor interface coupled with the sensor to preprocess a signal from the sensor; and a signal processing unit configured to convert the signal from the sensor to the processed signal that is readable by any CRM device that is capable of reading an electrocardiogram signal.

In some embodiments, the sensor interface preprocesses the signal from the sensor by buffering or amplifying the signal from the sensor. A network is configured to attenuate or amplify an output signal from the signal processing unit based on a prior or present input. The power source comprises pacing pulses from the CRM device. A switch may be closed to allow pacing pulses from the CRM device to reach cardiac tissue of a patient to which the lead is connected and is open when sensing of a physiological parameter of the patient is performed. The switch is controlled based on the signal from the sensor. The signal processing unit comprises an analog signal processing circuit, a digital signal processing circuit, or a passive signal processing circuit.

In accordance with another aspect of the invention, a method of processing a signal from a sensor coupled to a patient comprises receiving from the sensor a signal associated with a physiological parameter of a patient other than an electrocardiogram signal; converting the received signal associated with the physiological parameter other than the electrocardiogram signal into a converted signal that is readable by any CRM device that is capable of reading an electrocardiogram signal; and sending the converted signal to a CRM device.

In some embodiments, the converted signal is sent to the CRM device via a lead connecting the sensor to the CRM device. The received signal is converted by sensor electronics coupled to a lead connecting the sensor to the CRM device. The signal from the sensor is an analog signal or a digital signal. The method may further comprise sensing the physiological parameter of the patient by the sensor and transducing the sensed physiological parameter into the signal. Transducing may comprise modulating an amplitude and a frequency of the signal. Transducing may comprise pre-emphasizing the signal to compensate for a predefined electrocardiogram frequency response of the CRM device. Transducing may comprise attenuating the signal to an amplitude range of an electrocardiogram signal.

In specific embodiments, the method may further comprise performing DC restoration of the signal before sending the signal to the CRM device. It may further comprise compressing a time domain of the signal before sending the signal to the CRM device. It may further comprise encoding an offset which represents a type of the physiological parameter being sensed with the signal before sending the signal to the CRM device. It may further comprise adding a binary code which represents a type of the physiological parameter being sensed to the signal before sending the signal to the CRM device.

In some embodiments, the method further comprises powering the sensor and sensor electronics for converting the received signal which are coupled to a lead connecting the sensor to the CRM device. The sensor and the sensor electronics may be powered by pacing pulses from the CRM device. The sensor and the sensor electronics may be powered by a sensor battery located at the sensor. The sensor battery is recharged by pacing pulses from the CRM device. The sensor may be powered by a combination of chemicals (e.g., glucose and O2) obtained from the patient's body.

In accordance with another aspect of the present invention, a lead-based sensing system for use with a CRM comprises a sensor configured to be coupled to the CRM device by a lead, and to generate a signal associated with a physiological parameter of a patient; and sensor electronics connected to the lead to convert the sensed signal associated with the physiological parameter into a converted signal that is readable by the CRM device. The sensor and the sensor electronics are powered by pacing signals from the CRM device.

In some embodiments, the sensor is configured to sense a signal associated with a physiological parameter of a patient other than an electrocardiogram signal. The sensor electronics are configured to convert the sensed signal associated with the physiological parameter other than the electrocardiogram signal into a converted signal that is readable by any CRM device that is capable of reading an electrocardiogram signal. The sensor is configured to sense the physiological parameter of the patient and transduce the sensed physiological parameter into the signal. A sensor battery is located at the sensor for powering the sensor and the sensor electronics, and the sensor battery is recharged by pacing pulses from the CRM device. Alternatively, the battery and electronics can separated from the sensor and located near the CRM device.

### BRIEF DESCRIPTION OF THE DRAWINGS

FIG. 1 is a functional diagram of an illustrative embodiment of a CRM device/pacemaker system according to the present invention.

FIG. 2 is a flowchart illustrating the progression of a sensed signal through the CRM system.

FIG. 3 is a flowchart illustrating operation of the CRM system during pacing.

FIG. 4 is a more detailed diagram of an exemplary specialized lead.

FIG. 5 is a functional diagram of a pacing output stage from a CRM device.

FIG. 6 is a functional diagram of an exemplary sensor structure incorporated within a lead.

FIGS. 7A-7B is a diagram showing time concatenation of the received signal.

FIGS. 8A-8C is a diagram showing “chopped” playback from multiple signals.

FIGS. 9A-9C is a diagram showing alternated playback from multiple sensor streams.

FIG. 10 is a diagram showing a display signal on a programmer whereby the type of signal being sent and the units are displayed in conjunction with the signal.

FIG. 11 is a diagram showing DC restoration of the signal.

### DETAILED DESCRIPTION OF THE INVENTION

By creating a sensor lead structure that can interface with any CRM device through a conventional pacing/sensing port, medical practitioners are no longer restricted to using specialized CRM devices in conjunction with a specialized lead. Any CRM device can be used with the sensor lead structure described within this application. This affords the medical practitioner with a myriad of choices in the CRM devices available for implantation, thus reducing cost and increasing inter-compatibility between devices. Specialized leads can now be developed using the sensor lead structure described herein to be interfaced with any conventional CRM device.

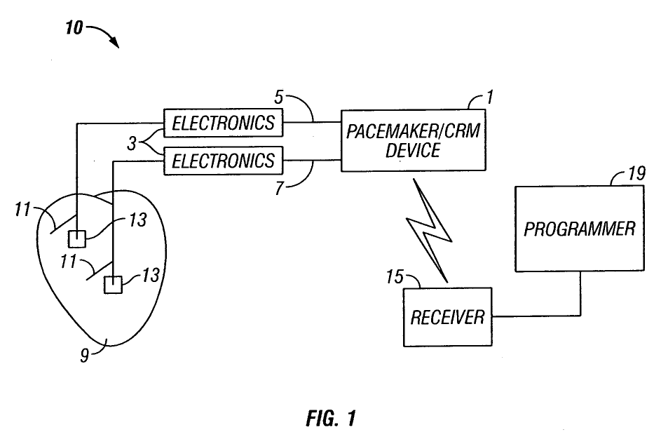

FIG. 1 is an exemplary diagram illustrating by way of example, but not by way of limitation an embodiment of a CRM device/pacemaker system 10 according to the present invention. A pacemaker or CRM device 1 that provides sensing of a specialized parameter (and optionally sensing of an electrocardiogram signal as well as pacing) is implanted within the body of patient at a location easily accessible to the patient's heart 9. Endocardial leads 5 and 7 are each coupled at a proximal end to the CRM device 1 through standard pacing/sensing ports, such as IS-1 compatible ports, and are each coupled at a distal end to the patient's heart 9 in first and second cardiac regions, respectively, that allow for electrical pacing impulses to be appropriately delivered to the heart 9 of the patient via ring electrode 77 and tip electrode 79 and also allow for sensing of physiological parameters via sensor 13 at the same or nearby locations. The body of the lead comprises a tubular sheath or housing made of an insulating, biocompatible, biostable material such as silicone rubber or polyurethane. The cross-sectional configuration of the lead body may accommodate various combinations of coil and/or cable conductor combinations including, for example, bipolar coaxial coils or bipolar cables or multilumen combinations of coils and/or cables. In one embodiment of the invention, a bipolar configuration can be implemented for the lead body where ring and tip electrodes are used as the conductive elements. One lead or many leads may be used in place of each of the two leads 5 and 7 shown in FIG. 1, depending on the number of physiological parameters to be sensed and whether a single-lead design is employed. The CRM device 1 selected may be from any manufacturer or model which provides a conventional pacing/sensing port for receiving signals that can be read by a conventional CRM device for processing electrocardiogram signals. The specific mention of an IS-1 compatible port is not meant to limit the invention, but is cited merely as an illustrative example of a conventional pacing/sensing port used in many current CRM devices. Additionally, while the leads are connected to an integrated pacing/sensing port on the CRM device, they can also be connected to separate pacing and sensing ports on the CRM device with no negative effects on the operation of the device.

Sensor electronics 3 are coupled to leads 7 and 9 in distal portions of the leads near the electrodes 11 and sensors 13. They provide signal processing and equalization of the received physiological parameters by processing the signal before it is sent back to the CRM device 1. The sensed signal is converted to a signal that can be read and processed by any CRM device, including a conventional CRM device that is configured to read and process electrocardiogram signals. Means of transformation include, for example, transmitting the signal as a pure analog signal, digitizing the signal, providing time contracted readback from one or multiple sensor signals, “chopping” multiple streams from the sensor together to form one output signal (i.e., alternating readback from multiple signals), DC restoration of the signal, or signal annotation. The sensor electronics can be powered by parasitically capturing energy obtained from the pacing pulses in an energy storage portion of the sensor circuitry, or the sensor electronics can be powered by an internal battery. Alternatively, pacing pulses can be used to recharge the internal battery if its charge level is low.

Once the physiological signal has been received at the CRM device 1, it can be stored for further retrieval within memory integrated within the CRM device. A number of different methods can be utilized to retrieve the signal received at the CRM device. One particular method of retrieval involves wirelessly transmitting information from the CRM device 1 to a wireless receiver 15. The information is then sent to a programmer 19, which may be coupled to the wireless transmitter 19, in a format easily readable by a medical practitioner or nurse. For example, when conventional sensing of a parameter such as a patient's heartbeat is performed, sensors 13 transform the physiological parameter being interpreted into an appropriate signal to be sent back to CRM device 1 over leads 5, 7 and that information is stored within memory at the CRM device 1 until retrieval is necessary by transmitting the stored signal to the wireless receiver 15 for interpretation by the programmer 19. While the process described above stores the physiological signal in memory for future retrieval, the CRM system 10 can also display the signal being measured in real-time, dependent only on the latency of the wireless link between the CRM device 1 and the wireless receiver 15.

Alternation between pacing and sensing occurs within the sensor electronics when a switch provided within the sensor electronics actively drives two possible signal paths, depending on whether data collection or voltage through the lead is needed corresponding to a surrogate of the electrocardiogram signal.

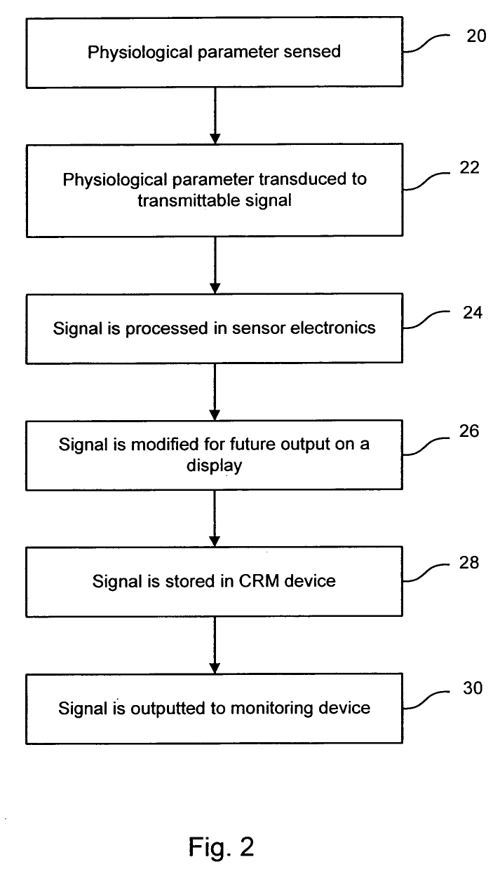

An embodiment of the sensing process is shown in FIG. 2, which is a flowchart illustrating the progression of a sensed signal through the CRM system 10. In step 20, a physical parameter (e.g., pressure, temperature, pH, displacement, acceleration, voltage, current, frequency, period, strain, force, acoustical parameters, fluid flow rate, and blood-oxygen saturation) is traced by the sensors 13 within the body of the patient. The physiological parameter is transduced from the parameter being read to a transmittable signal by the sensors 13 in step 22. The parameter being measured can then be attenuated to the amplitude range typical of an electrocardiogram signal or pre-emphasized to compensate for the electrocardiogram frequency response. The signal can then be processed into a more appropriate signal to be sent back to the CRM device by the sensor electronics 3 in step 24. These signal processing steps may include, for example, transmitting the signal as a pure analog signal, digitizing the signal, providing time contracted readback from one or multiple sensor signals, “chopping” multiple streams from the sensor together to form one output signal, alternating readback from multiple signals, DC restoration of the signal, or other similar processes, performed alone or in combination. The transduced signal can also be manipulated in step 26 to be normalized for proper display of units on an external programmer by signal annotation or auto-signaling the type to the CRM device within transmission of the signal. Auto-signaling may be performed by sending a machine readable code that may be hidden in a blank portion of the signal, transmitted as a low-level signal, or sent as a burst signal. Alternatively, signal annotation may be used whereby the signal will be a human readable form such as a character that is imposed periodically in conjunction with the sensor signal to visually represent the type of sensor on an electrocardiogram-type sensing structure. Similarly, a calibration signal may be imposed for reference purposes or as a means of DC restoration to indicate absolute signals either by “chopping” at a low duty cycle or chopping at a high rate. DC restoration is of particular use when communicating a slowly-changing input such as, for example, temperature, where removal of the DC bias component can lead to an improper reading of the signal. By setting a known reference level that is communicated to the output device, the physiological parameter can be measured in terms of its relative offset to the reference level. The signal is then sent to the CRM device through the leads 5, 7 and stored in memory located in the CRM device in step 28. When review of the signal is needed, the signal is outputted from the CRM device to a wireless monitoring device and displayed in step 30.

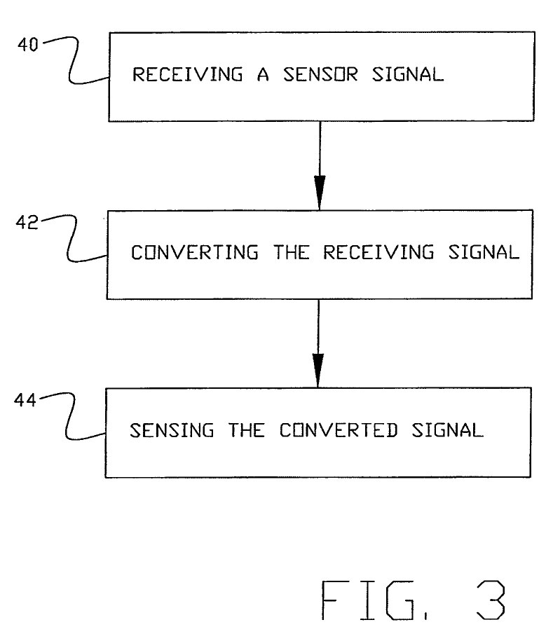

FIG. 3 is a flowchart illustrating an exemplary method. Block 40 in FIG. 3 illustrates the step of receiving from a sensor a signal associated with a physiological parameter of a patient (other than an electrocardiogram signal). Block 42 illustrates the step of converting the received signal associated with the physiological parameter other than the electrocardiogram signal into a converted signal that is readable by any CRM device that is capable of reading an electrocardiogram signal. Block 44 illustrates the step of sending the converted signal to a CRM device.

A more detailed example of an exemplary specialized lead will be discussed in relation to FIG. 4, which shows a sensor adapted to continuously monitor intracardiac pressures sensed through a pressure transfer tube. The lead can be implanted in the right ventricular outflow tract. The entire lead 54 is housed within a tubular sheath or housing made of a sterile material such as silicone rubber or polyurethane to prevent unexpected reactions or inflammation during implantation. The right end 56 of the cylindrical housing has a pressure transfer tube to measure tube-end pressure which is coupled with a measuring unit 48. The measuring unit 48 measures the captured pressure and accurately outputs its value to the sensor electronics 3, which will be described in more detail below. The output of the sensor electronics 3 is the signal that is sent through the remainder of the lead to the CRM device 1 to be stored for retrieval.

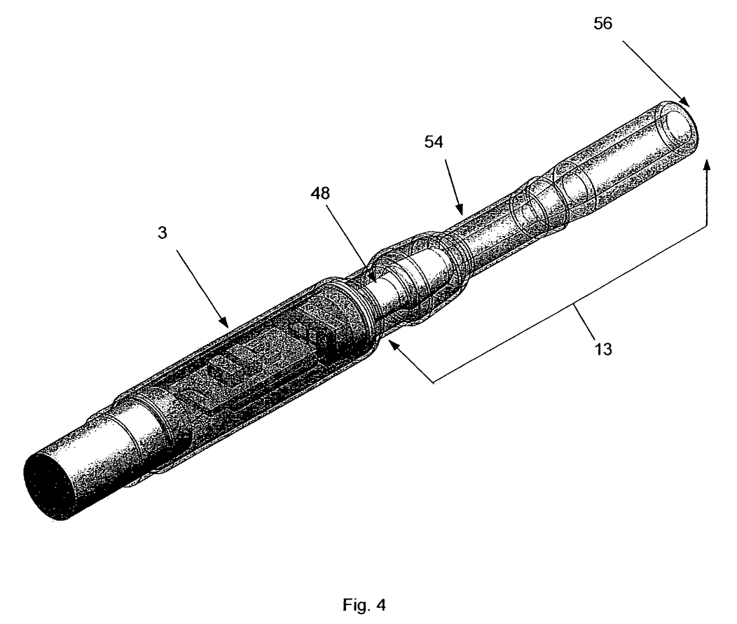

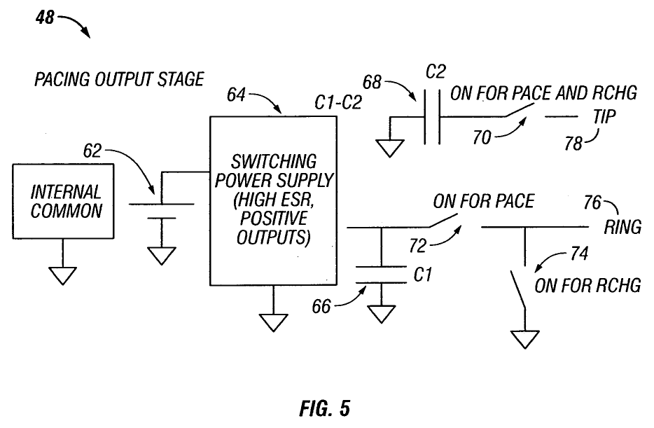

FIG. 5 shows a functional diagram of a pacing output stage of a CRM device. This pacing output stage is located within the CRM device itself, in proximity to the sensing/pacing ports of the device. A battery 62 supplies the pacing output of the circuit, and is fed into a switching power supply 64. The switching power supply 64 is present to regulate voltage output from the battery 62 to an amount consistent with an appropriate pacing output. The switching power supply 64 possesses a high effective series resistance and outputs a positive voltage. A capacitor (C1) 66 is connected to the internal common voltage and is charged by the difference in voltage between the switching power supply 64 and the internal common. A switch 72 is closed when pacing takes place to create a direct path for the output voltage to be sent from a ring electrode contact 76 for stimulation of the cardiac tissue. A switch 74 is closed when recharging of the sensor electronics occurs. A capacitor (C2) 68 is connected to the internal common on one side and to a tip electrode contact 78.

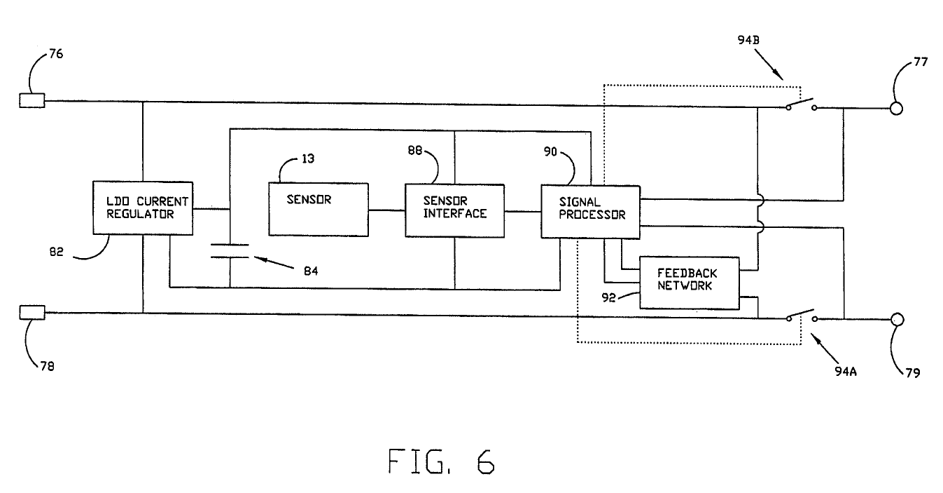

FIG. 6 shows an example of pacing powered sensor electronics integrated into a lead structure. The ring electrode contact 76 and the tip electrode contact 78 are connected to an LDO current regulator 82 to provide the sensor electronics with a constant source of reliable power to ensure accurate processing of the physiological parameters being measured. A limited amount of current is taken from the pacing pulses and stored within a capacitor 84, so as not to inhibit the stimulation of cardiac tissue if pacing is needed. In other words, the sensor electronics can be powered by parasitically drawing energy from the pacing pulses sent from the CRM device, without adversely affecting the pulses being sent. Even if no pacing is required due to sensor measurements, pacing pulses can be diverted to power the sensor electronics with no voltage being sent to the patient's cardiac tissue. Thus, the power for the internal electronics is rectified, stored and regulated for use with the sensor electronics. When a pacing pulse is sent from the CRM device, it enters via the ring electrode contact 76 and the tip electrode contact 78 and proceeds through switches 94A and 94B which are closed during pacing, through ring electrode 77 and tip electrode 79, to the cardiac tissue. Sensor 11 is connected to a sensor interface 88, which performs buffering or amplification of the transduced output signal. The capacitor 84 is used to power the sensor interface 88 and the signal processing unit 90. The output signal is then passed to the signal processing unit 90, which can be implemented as a digital, analog, or passive processing unit depending on the specific requirements of the device. The signal processing device 90 functions as the control means at the end of the sensor that makes the interface of raw data usable to the CRM device. A wide variety of functions can be implemented alone or in combination within the signal processing unit 90. The output of the signal processing unit 90 leads to a feedback network 92, which attenuates or amplifies the output signal from the signal processing unit 90 based on prior or present inputs. Additionally, switches 94A and 94B are incorporated in the lead near the contact points to be opened during sensing when pacing is not needed, and closed during pacing when an electrical connection is needed. A tri-state mode system may be implemented in the system which switches between three modes high, low, and off, depending on whether pacing is needed. The processing signal is then sent back over the ring electrode contact 76 and tip electrode contact 78 to the CRM device 1 to be stored or displayed for further output. After the signal has been processed, it can be sent to the wireless transmitter 15 and the programmer 19 for immediate real-time playback, or can be stored in the CRM device 1 for playback at a later time.

Another reason signal processing is necessary is that conventional CRM devices are designed to only display an electrocardiogram signal and do not have the capability to handle or display multiple sensor inputs or display a signal different from an electrocardiogram signal. Thus, care must be taken to process the signal being measured by a specialized lead so that it can be viewed on a conventional CRM device. As detailed previously, these methods include, for example, pre-emphasizing the signal to compensate for the electrocardiogram frequency response of a CRM device, attenuating the signal to an amplitude range typical of an electrocardiogram signal, or any of the methods of signal processing described below. A second problem that can occur is when multiple parameters are being monitored by a group of specialized leads, and the CRM device is programmed to display a single electrocardiogram signal. Different signal processing methodologies such as signal “chopping” or time concatenation described in more detail can help alleviate this problem. Another problem that can occur when connecting specialized leads to a conventional CRM device is that the captured signals must be normalized for proper display of units on an external programmer. Steps can be taken to either auto-signal the lead interface type to the CRM device or to apply a method of signal annotation where the type of signal being measured is displayed on an viewing device.

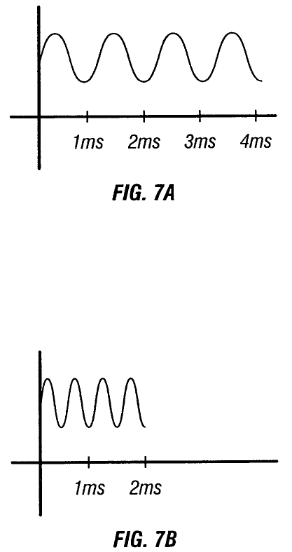

FIGS. 7A-7B shows an example of a type of signal processing performed in the signal processing unit 90 before the output signal is sent to the CRM device. FIG. 7A shows time concatenation of a signal or time contracted playback of the signal at the CRM device. The signal entering the signal processing unit is shown in FIG. 7A, and the output is shown in FIG. 7B. The signal in 7A has been compressed into a shorter time period to help accommodate for latency in the readback of the signal, allowing the information to be stored or displayed in a more efficient manner. This method may be particularly useful when applied to multiple input signals, allowing for the multiple signals to be displayed in a shorter timeframe. Multiple channels from the sensors can be read simultaneously, stored in local memory, and played back at an accelerated rate afterwards.

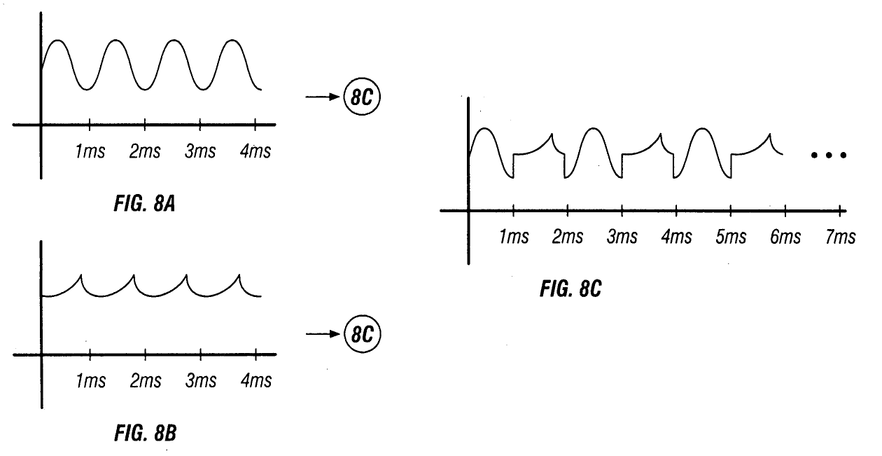

FIGS. 8A-8C present another example of a type of signal processing that shows “chopped” or alternating playback from two sensor streams. FIGS. 8A and 8B show two sensor streams being received, and FIG. 8C shows the two signals being viewed alternately, one signal for 1 ms, and the other signal for the next 1 ms, and so on. By switching between the two signals, any significant distortion or deterioration of the parameter being monitored can be easily seen without having to switch between the signals, if that capability is available in the CRM device. Chopped playback allows for two or more sensor streams to be displayed concurrently on the display output of the CRM system.

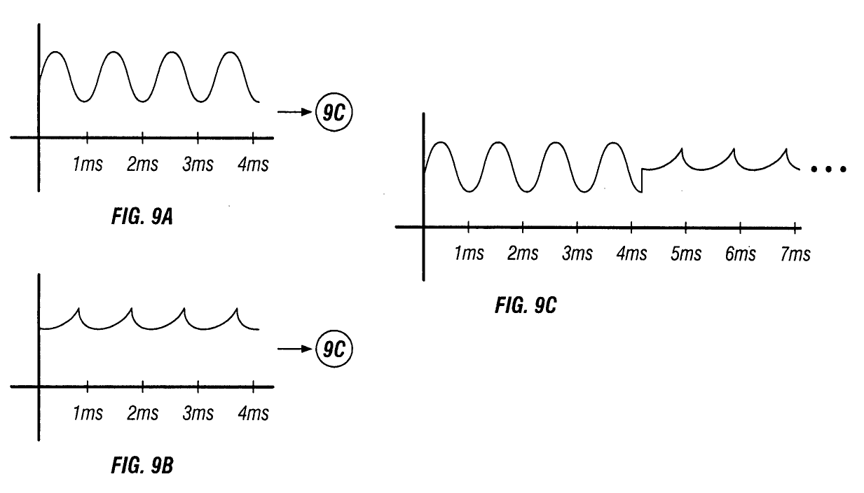

FIGS. 9A-9C show a form of signal processing utilizing alternating playback between different sensor signals. Two input signals are shown in FIGS. 9A and 9B, which are combined in an alternating sequence in FIG. 9C. One signal can be played back in its entirety, then the other signal played back afterwards. Alternatively, selected portions of the input signals may be played back in an alternating manner. While only two input signals are shown within the FIGS. 9A and 9B, it is understood that the technique can apply to multiple sensor streams more than two as well. Additionally, a priority system may be implemented within the sensor electronics to display input signals based on an assigned priority value for each input signal during playback.

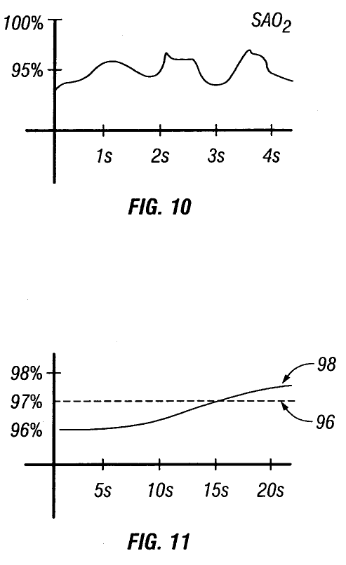

FIG. 10 shows an example of a display signal on a programmer whereby the type of signal being sent and the units are displayed in conjunction with the signal. The signal being monitored is “SAO₂,” or blood-oxygen saturation, and the appropriate units are conveyed in conjunction with the signal for correct display and storage. This can be performed by the lead interface auto-signaling its type to the CRM device by sending a machine readable code that may be hidden in a blank portion of the signal, transmitted as a low-level or high level signal, sent as a burst signal, or encoded as an offset signal. This auto-signaling can be sent before the actual signal as a header, or interleaved with the signal in a concurrent manner. Alternatively, signal annotation can be used whereby the signal will be a human readable form such as a character that is imposed periodically in conjunction with the sensor signal to visually represent the type of sensor on an electrocardiogram-type sensing structure.

Another problem that can occur when displaying a signal is that the CRM device only can accept a certain range of signals as being input. The signal processing unit can change the profile of the signals being sent to the CRM device to an inverse function to compensate for the bandpass characteristics of the CRM device.

FIG. 11 shows an example of DC restoration of a signal at the CRM device or programmer. Conventional CRM devices use AC-coupled inputs to block the DC component of a signal but extract the AC component of the signal. One problem that occurs with this process is that it makes conveying a slowly changing input parameter such as temperature difficult in that gradual changes cannot be displayed accurately. The solution to this is to use a DC restoration process whereby a known reference level is communicated in conjunction with the signal which establishes a reference point at which all other signals are set. The reference level can be set and established prior to transmission of the signal for display or storage and the signal interpreted in the context of the reference level. In the drawing, reference level 96 is used to interpret the signal 98 and determine the units being used.

While embodiments of the invention have been described which utilize pacing pulses to power the sensor electronics, alterative powering methods may be used as well. For example, pacing is not needed during intrinsic cardiac activity, so no power is provided. Enough energy can be stored within the device to power it over at least one cardiac cycles, or enough energy can be stored plus a delay period implemented so that the sensors power up after hemodynamic stability is reached without pacing. Alternatively, the sensor can be powered through a special independently paced port, or an internal battery can be used to power the sensing device and the pacing pulses used when available to recharge the battery. In another embodiment of the invention, a power system may be implemented within the lead which absorbs chemicals (e.g., glucose and O2) from the body for use as a battery and being used to power the sensor electronics.

It is to be understood that the above description is intended to be illustrative and not restrictive. Many embodiments will be apparent to those of skill in the art upon reviewing the above description. The scope of the invention should, therefore, be determined not with reference to the above description, but instead should be determined with reference to the appended claims alone with their full scope of equivalents.

What is claimed is:

1. A lead-based sensing system for use with any cardiac rhythm management (CRM) device that is capable of reading an electrocardiogram signal, the lead-based sensing system comprising:
a sensor configured to be coupled to any CRM device by a lead, and to generate a signal associated with a physiological parameter of a patient other than an electrocardiogram signal; and
sensor electronics connected to the lead to convert the signal associated with the physiological parameter other than the electrocardiogram signal into a converted signal that is readable by any CRM device that is capable of reading an electrocardiogram signal.

2. The lead-based sensor system of claim 1 wherein the sensor is configured to sense a signal associated with any of pressure, temperature, pH, displacement, acceleration, voltage, current, frequency, period, strain, force, acoustical parameters, fluid flow rate, and blood-oxygen saturation. %%

3. The lead-based sensor system of claim 1 wherein the sensor electronics comprise:
a sensor interface coupled with the sensor to pre-process the signal from the sensor; and
a signal processing unit configured to convert the signal from the sensor to the processed signal that is readable by any CRM device that is capable of reading an electrocardiogram signal.

4. The lead-based sensor system of claim 3 wherein the sensor electronics further comprise:
a current regulator connected to a power source which regulates output voltages to be between minimum and maximum levels; and
a capacitor coupled to the current regulator to store an electrical charge and power a circuit comprising the sensor electronics.

5. The lead-based sensor system of claim 4 wherein the power source comprises pacing pulses from the CRM device.

6. A circuit for interfacing a sensor to a lead for a cardiac rhythm management (CRM) device, the circuit comprising:
a current regulator connected to a power source which regulates output voltages to be between minimum and maximum levels;
a capacitor coupled to the current regulator to store an electrical charge and power the circuit;
a sensor interface coupled with the sensor to preprocess a signal from the sensor; and
a signal processing unit configured to convert the signal from the sensor to the processed signal that is readable by any CRM device that is capable of reading an electrocardiogram signal.

7. The circuit of claim 6 wherein the sensor interface preprocesses the signal from the sensor by buffering or amplifying the signal from the sensor.

8. The circuit of claim 6 further comprising a feedback network configured to attenuate or amplify an output signal from the signal processing unit based on a prior or present input.

9. The circuit of claim 6 wherein the power source comprises pacing pulses from the CRM device.

10. The circuit of claim 6 further comprising a switch which is closed to allow pacing pulses from the CRM device to reach cardiac tissue of a patient to which the lead is connected and is open when sensing of a physiological parameter of the patient is performed.

11. The circuit of claim 10 wherein the switch is controlled based on the signal from the sensor.

12. The circuit of claim 6 wherein the signal processing unit comprises an analog signal processing circuit, a digital signal processing circuit, or a passive signal processing circuit.

13. A method of processing a signal from a sensor coupled to a patient, the method comprising:
receiving from the sensor a signal associated with a physiological parameter of a patient other than an electrocardiogram signal;
converting the received signal associated with the physiological parameter other than the electrocardiogram signal into a converted signal that is readable by any CRM device that is capable of reading an electrocardiogram signal; and
sending the converted signal to a CRM device.

14. The method of claim 13 wherein the converted signal is sent to the CRM device via a lead connecting the sensor to the CRM device.

15. The method of claim 13 wherein the received signal is converted by sensor electronics coupled to a lead connecting the sensor to the CRM device.

16. The method of claim 13 wherein the signal from the sensor is an analog signal or a digital signal.

17. The method of claim 13 further comprising sensing the physiological parameter of the patient by the sensor and transducing the sensed physiological parameter into the signal.

18. The method of claim 17 wherein transducing comprises modulating an amplitude and a frequency of the signal.

19. The method of claim 17 wherein transducing comprises pre-emphasizing the signal to compensate for a predefined electrocardiogram frequency response of the CRM device.

20. The method of claim 17 wherein transducing comprises scaling the signal to an amplitude range of an electrocardiogram signal.

21. The method of claim 13 further comprising performing DC restoration of the signal before sending the signal to the CRM device.

22. The method of claim 13 further comprising compressing a time domain of the signal before sending the signal to the CRM device.

23. The method of claim 13 further comprising encoding an offset which represents a type of the physiological parameter being sensed with the signal before sending the signal to the CRM device.

24. The method of claim 13 further comprising adding a binary code which represents a type of the physiological parameter being sensed to the signal before sending the signal to the CRM device.

25. The method of claim 13 further comprising powering the sensor and sensor electronics for converting the received signal which are coupled to a lead connecting the sensor to the CRM device.

26. The method of claim 25 wherein the sensor and the sensor electronics are powered by pacing pulses from the CRM device.

27. The method of claim 25 wherein the sensor and the sensor electronics are powered by a sensor battery located at the sensor.

28. The method of claim 27 wherein the sensor battery is recharged by pacing pulses from the CRM device.

29. The method of claim 13 wherein the sensor is powered by a combination of chemicals obtained from the patient's body.

30. A lead-based sensing system for use with a cardiac rhythm management (CRM), the lead-based sensing system comprising:
a sensor configured to be coupled to the CRM device by a lead, and to generate a signal associated with a physiological parameter of a patient; and
sensor electronics connected to the lead to convert the sensed signal associated with the physiological parameter into a converted signal that is readable by the CRM device;
wherein the sensor and the sensor electronics are powered by pacing signals from the CRM device.

31. The lead-based sensing system of claim 30 wherein the sensor is configured to sense a signal associated with a physiological parameter of a patient other than an electrocardiogram signal; and wherein the sensor electronics are configured to convert the sensed signal associated with the physiological parameter other than the electrocardiogram signal into a converted signal that is readable by any CRM device that is capable of reading an electrocardiogram signal.

32. The lead-based sensor system of claim 30 wherein the sensor is configured to sense the physiological parameter of the patient and transduce the sensed physiological parameter into the signal.

33. The lead-based sensor system of claim 30 further comprising a sensor battery located at the sensor for powering the sensor and the sensor electronics, wherein the sensor battery is recharged by pacing pulses from the CRM device.
